(19) United States
(12) Patent Application Publication
(10) Pub. No.: US 2025/0352316 A1
(43) Pub. Date: Nov. 20, 2025

(54) CONDUCTIVE STRUCTURE OF SONIC MOTOR, ELECTRIC TOOTHBRUSH AND BRUSH HEAD POWER SUPPLY STRUCTURE THEREFOR

(71) Applicants: BIXDO (SH) HEALTHCARE TECHNOLOGY CO., LTD., Shanghai (CN); GUANGDONG BIXDO HEALTH TECHNOLOGY CO., LTD., Foshan (CN)

(72) Inventors: Zhongping HAN, Foshan (CN); Dongdong QIAN, Foshan (CN); Xuefeng WANG, Foshan (CN); Yanbo ZHANG, Foshan (CN); Jun PENG, Foshan (CN)

(21) Appl. No.: 19/283,429

(22) Filed: Jul. 29, 2025

Related U.S. Application Data
(63) Continuation of application No. PCT/CN2024/130346, filed on Nov. 6, 2024.

(30) Foreign Application Priority Data
Nov. 20, 2023 (CN) 202311552044.0

(51) Int. Cl. A61C 17/22; A46D 1/00; A61C 17/34

(52) U.S. Cl. CPC A61C 17/222; A46D 1/02; A61C 17/3481

(57) ABSTRACT

A conductive structure of a sonic motor, an electric toothbrush and a brush head power supply structure therefor. The structure includes a sonic motor body, a hollow conductive motor shaft and a conductive assembly. The shaft is a hollow conductive structure. An interior of the shaft is a wiring channel. The conductive assembly passes through and out of the body from the shaft along an axis of the motor shaft. The shaft itself can be used as a second conducting path. Both ends of each of the two conducting paths are connected to the power-consuming component and a power supply respectively, form an electrically conductive path. The characteristics of the structure of the sonic motor itself is fully utilized. Only the motor shaft needs to be set to be hollow, and there is no need for a significant structural modification to the motor.

### CROSS-REFERENCE TO RELATED APPLICATION

This application is a continuation of International Application No. PCT/CN2024/130346, filed on Nov. 6, 2024, which claims priority to and benefits from Chinese Patent Application No. 202311552044.0 filed with the CNIPA on Nov. 20, 2023, the entire contents of both of which are incorporated herein by reference.

### FIELD

The present application relates to the field of electric toothbrushes, and more particularly, to a conductive structure of a sonic motor, an electric toothbrush and a brush head power supply structure therefor.

### BACKGROUND

With the development of social intelligence, more and more electrical appliances have appeared in people's lives. In addition, with the improvement of living standards, more and more consumers pay attention to oral health issues. For example, an electric toothbrush as a type of electrical appliances, utilizes a sonic motor as a driver to generate power, thereby creating high-frequency reciprocating vibrations with a certain amplitude so as to drive the brush head for high-frequency swing, and thus achieving teeth cleaning and greatly facilitating daily life. At present, there are relatively few electric toothbrushes on the market in which the brush head is luminous and has additional functions, most electric toothbrushes are conventional electric toothbrushes. A few blue or red light electric toothbrushes often require a significant change to a motor output shaft to complete the power supply wiring so as to supply power to the brush heads, which makes the structures complex. Alternatively, due to the increase in number of components or the change in motor type, the motion transmission efficiency may suffer a significant loss, causing the swing amplitude and frequency of brush heads to be reduced. For example, a Chinese Patent CN215019476U discloses a structure of a sonic electric toothbrush with sterilization, disinfection and blue light whitening functions, and the sonic electric toothbrush accomplishes power supply through wireless charging coils, causing an increase in the number of components and resulting in an unsatisfactory effect of the brush head swing. Alternatively, in order to accommodate conductive devices, a volume of the brush head and the motor output shaft is increased, thereby reducing the user experience. Based above, the present disclosure has been developed.

### SUMMARY

In view of the above, according to one aspect, the present disclosure aims to solve the problem that the power supply structure of the sonic motor in the electric toothbrushes on the market is relatively complex, a significant change to the motor output shaft is required and would affect the motion transmission efficiency.

According to another aspect, the present disclosure aims to solve the problem that the power supply structure of the sonic motor in some electric toothbrushes in the market has a complex wiring arrangement, causing assembling and mounting can not be achieved.

According to yet another aspect, the present disclosure aims to solve the problem that in some electric toothbrushes in the market, the sonic motor requires change in the spatial structure of the motor output shaft or brush head to accommodate the conductive components.

To solve one of the above problems, the present disclosure provides a conductive structure of a sonic motor. The conductive structure includes a sonic motor body, a hollow conductive motor shaft, a conductive assembly and a mounting bracket. One end of the hollow conductive motor shaft passes through an interior of the sonic motor body and is driven by the sonic motor body to perform high-frequency reciprocating vibrations with a certain amplitude. A segment of the hollow conductive motor shaft extends out from the sonic motor body to form an output segment. The output segment has a first electrical output terminal. The hollow conductive motor shaft has a first electrical input terminal. The conductive assembly at least includes a first conductive component, a second conductive component and an insulating mounting base. The first conductive component is installed on the output segment via the insulating mounting base to form a second electrical output terminal. One end of the second conductive component is connected to the first conductive component, another end of the second conductive component passes through the hollow conductive motor shaft and extends out from the hollow conductive motor shaft to form a second electrical input terminal. The mounting bracket is sleeved on the hollow conductive motor shaft and is at least circumferentially fixed relative to the hollow conductive motor shaft. The mounting bracket has a first mounting port at the first electrical output terminal for mounting and connecting a power-consuming component. The mounting bracket has a second mounting port at the second electrical output terminal for mounting and connecting the power-consuming component.

Preferably, the hollow conductive motor shaft further includes a motor shaft wire. One end of the motor shaft wire is in electrical connection with the first electrical output terminal, and another end of the motor shaft wire is implemented as the first electrical input terminal.

Preferably, a connection hole is formed on a side wall of the output segment. The one end of the motor shaft wire is connected to the hollow conductive motor shaft in a hidden way via the connection hole, and the other end of the motor shaft wire extends within the hollow conductive motor shaft or extends outside the hollow conductive motor shaft.

Preferably, the conductive structure further includes a first conductive plate. The first conductive plate has a through hole. The hollow conductive motor shaft passes through the through hole and is in contact with the first conductive plate. The one end of the motor shaft wire is connected to the first conductive plate.

Preferably, the first conductive plate is further provided with a flexible contact portion in the through hole. The first conductive plate is electrical contact with the hollow conductive motor shaft via the flexible contact portion.

Preferably, the first conductive component is a first electrode. The second conductive component is a second wire. The first electrode is installed at a rear end of the output segment via the insulating mounting base. The second wire passes through the hollow conductive motor shaft and is connected to the first electrode. The second electrical input terminal is defined as an end of the second wire that is far away from the first electrode. The second electrical output terminal is defined as the first electrode.

Preferably, the conductive structure further includes an electrical connection assembly. The electrical connection assembly includes a first electrical connector and a second electrical connector. The first electrical connector is detachably mounted at the first mounting port and is in contact with the first electrical output terminal. The second electrical connector is detachably mounted at the second mounting port and is in contact with the second electrical output terminal. The first electrical connector and the second electrical connector are connected to positive and negative electrodes of the power-consuming component through wires to provide power.

Beneficial effects of the above technical solutions may result from any one of the following items or any combination thereof

In the present disclosure, the motor shaft of the sonic motor is implemented as a hollow conductive structure. An interior of the motor shaft is implemented as a wiring channel. The conductive assembly passes through and out of the body from the motor shaft along an axis of the motor shaft for connecting to an element, so as to form a first conducting path. The motor shaft itself can be used as a second conducting path due to its conductive property. Both ends of each of the two conducting paths are connected to the power-consuming component and a power supply respectively, so as to form an electrically conductive path. The characteristics of the structure of the sonic motor itself is fully utilized. Only the motor shaft needs to be set to be hollow, and there is no need for a significant structural modification to the motor. Moreover, both the first electrical output terminal and the second electrical output terminal are integrated with the motor shaft itself, thus the back-and-forth vibrations of the motor at a high frequency does not affect the electrical connection efficiency and no extra wiring layout is required. The two output terminals can be contact with corresponding inner parts stably via the mounting bracket and can be mounted with an external part in an assembled way, and thus has incomparable advantages in both security and convenience.

The external output linkage is completed through the mounting bracket. At the same time, due to a structure of the mounting bracket, the power-consuming component can be installed on the mounting bracket and can be assembled with the mounting ports on the mounting bracket, so that electrical connection of the two output terminals can be achieved.

Wire paths can be completely routed inside the hollow conductive motor shaft, or one wire path can be led out from the hollow conductive motor shaft via the motor shaft wire to connect to an external element. In this way, the present application has a strong adaptability and a wide range of application. It is particularly suitable for a conductive structure in which the power supply is located on one side of the sonic motor body and the power-consuming component is located on the other side of the sonic motor body. It has a strong integration, a high space utilization and low difficulty of arranging wires, and improves the durability.

The two electrical output terminals are connected through the electrical connection assembly in an assembled way, and the assembling is very convenient. The conducting paths are clear. The conducting path of the power supply structure is integrated on the sonic motor, and the conducting path of the power-consuming component is integrated into the structure of the power-consuming component. The conducting path of the power supply structure and the conducting path of the power-consuming component only need to be assembled and installed to contact with each other. The mounting bracket further stabilizes the contact structure between the two conducting paths. Compared with the pure wire connection available on the market, reliability in the present application is greatly improved.

To solve one of the problems, the present disclosure provide a brush head power supply structure for an electric toothbrush. The brush head power supply structure for an electric toothbrush includes at least a brush head and the conductive structure of the sonic motor as described above. The power-consuming component is arranged in the brush head. A positive electrode and a negative electrode of the power-consuming component are correspondingly connected to the first electrical output terminal and the second electrical output terminal.

Preferably, The brush head power supply structure further includes a brush head handle, and the brush head is arranged on the brush head handle. The brush head handle has an insertion cavity. An electrical connection assembly is provided in the insertion cavity. The electrical connection assembly includes a first electrical connector and a second electrical connector, the first electrical connector and the second electrical connector are respectively connected to the positive electrode and the negative electrode of the power-consuming component through wires. The mounting bracket is inserted into the insertion cavity. The first electrical connector is detachably mounted at the first mounting port and is in contact with the first electrical output terminal, the second electrical connector is detachably mounted at the second mounting port and is in contact with the second electrical output terminal.

Preferably, the first electrical connector is conductive elastic plate. An elastic deformation portion is provided at a bottom of the conductive elastic plate. A guide slot is provided on the mounting bracket along an insertion direction. The first mounting port is located in the guide slot. The conductive elastic plate compresses the elastic deformation portion to allow the elastic deformation portion to be inserted into the first mounting port along the guide slot. The elastic deformation portion extends into the first mounting port and is in electrical contact with the first electrical output terminal.

Preferably, The second electrical connector is a conductive probe. The conductive probe is inserted into the second mounting port and is in electrical contact with the first conductive component.

Beneficial effects of the above technical solutions may result from any one of the following items or any combination thereof.

In the present disclosure, the motor shaft of the sonic motor is implemented as a hollow conductive structure. An interior of the motor shaft is implemented as a wiring channel. The conductive assembly passes through and out of the body from the motor shaft along an axis of the motor shaft for connecting to an element, so as to form a first conducting path. The motor shaft itself can be used as a second conducting path due to its conductive property. Both ends of each of the two conducting paths are connected to the power-consuming component and a power supply respectively, so as to form an electrically conductive path. The characteristics of the structure of the sonic motor itself is fully utilized. Only the motor shaft needs to be set to be hollow, and there is no need for a significant structural modification to the motor. Moreover, both the first electrical output terminal and the second electrical output terminal are integrated with the motor shaft itself, thus the back-and-forth vibrations of the motor at a high frequency does not affect the electrical connection efficiency and no extra wiring layout is required. The two output terminals can be contact with corresponding inner parts stably via the mounting bracket and can be mounted with an external part in an assembled way and thus has incomparable advantages in both security and convenience.

The external output linkage is completed through the mounting bracket. At the same time, due to a structure of the mounting bracket, the power-consuming component can be installed on the mounting bracket by being supported by a structure of the mounting bracket and can be assembled with the mounting ports on the mounting bracket, so that electrical connection of the two output terminals can be achieved.

Wire paths can be completely routed inside the hollow conductive motor shaft, or one wire path can be led out from the hollow conductive motor shaft via the motor shaft wire to connect to an external element. In this way, the present application has a strong adaptability and a wide range of application. It is particularly suitable for a conductive structure in which the power supply is located on one side of the sonic motor body and the power-consuming component is located on the other side of the sonic motor body. It has a strong integration, a high space utilization and low difficulty of arranging wires, and improves the durability.

The two electrical output terminals are assembled and connected through the electrical connection assembly in an assembled way, and the assembling is very convenient. The conducting paths are clear. The conducting path of the power supply structure is integrated on the sonic motor, and the conducting path of the power-consuming component is integrated into the structure of the power-consuming component. The conducting path of the power supply structure and the conducting path of the power-consuming component only need to be assembled and installed to contact with each other. The mounting bracket further stabilizes the contact structure between two conducting paths. Compared with the pure wire connection available on the market, reliability in the present application is greatly improved.

The mounting bracket is inserted into the brush head handle in the electric toothbrush, so that the sonic motor body electromagnetically drives the hollow conductive motor shaft and thus the hollow conductive motor shaft drives the brush head handle to rotate reciprocally at a high-frequency through the mounting bracket. Finally, the brush head is driven to swing at a high frequency. The two electrical connectors are implemented as the conductive probe and conductive elastic plate respectively. The conductive probe is inserted into the first electrode. The conductive elastic plate is assembled with the mounting bracket via the guide slot. The structure of the mounting bracket achieves stable electrical contact, thereby achieving a high assembly performance and a good stability.

To solve one of the above problems, the present disclosure further provides an electric toothbrush. The electric toothbrush includes the brush head power supply structure for an electric toothbrush as described above. The first electrical input terminal and the second electrical input terminal are connected to a control main board or a battery of the electric toothbrush.

Preferably, The power-consuming component is a lighting effect function component. The lighting effect function component includes a light board and a light bead. The light board is installed in the brush head. The light board is electrically connected to the first electrical output terminal and the second electrical output terminal. The light bead is installed on the light board.

Preferably, the light bead is implemented as one of a red light therapy bead, a blue light brightening bead or a violet light sterilization bead or any combination thereof.

Preferably, bristles of the electric toothbrush are partially implemented as light-conducting bristles.

Preferably, the power-consuming component is implemented as a sensor and/or a photographic component.

Beneficial effects of the above technical solutions may result from any one of the following items or any combination thereof.

In the present disclosure, the motor shaft of the sonic motor is implemented as a hollow conductive structure. An interior of the motor shaft is implemented as a wiring channel. The conductive assembly passes through and out of the body from the motor shaft along an axis of the motor shaft for connecting to an element, so as to form a first conducting path. The motor shaft itself can be used as a second conducting path due to its conductive property. Both ends of each of the two conducting paths are connected to the power-consuming component and a power supply respectively, so as to form an electrically conductive path. The characteristics of the structure of the sonic motor itself is fully utilized. Only the motor shaft needs to be set to be hollow, and there is no need for a significant structural modification to the motor. Moreover, both the first electrical output terminal and the second electrical output terminal are integrated with the motor shaft itself, thus the back-and-forth vibrations of the motor at a high frequency does not affect the electrical connection efficiency and no extra wiring layout is required. The two output terminals can be contact with corresponding inner parts stably via the mounting bracket and can be mounted with an external part in an assembled way, and thus has incomparable advantages in both security and convenience.

The external output linkage is completed through the mounting bracket. At the same time, due to a structure of the mounting bracket, the power-consuming component can be installed on the mounting bracket and can be assembled with the mounting ports on the mounting bracket, so that electrical connection of the two output terminals can be achieved.

Wire paths can be completely routed inside the hollow conductive motor shaft, or one wire path can be led out from the hollow conductive motor shaft via the motor shaft wire to connect to an external element. In this way, the present application has a strong adaptability and a wide range of application. It is particularly suitable for a conductive structure in which the power supply is located on one side of the sonic motor body and the power-consuming component is located on the other side of the sonic motor body. It has a strong integration, a high space utilization and low difficulty of arranging wires, and improves the durability.

The two electrical output terminals are connected through the electrical connection assembly in an assembled way, and the assembling is very convenient. The conducting paths are clear. The conducting path of the power supply structure is integrated on the sonic motor, and the conducting path of the power-consuming component is integrated into the structure of the power-consuming component. The conducting path of the power supply structure and the conducting path of the power-consuming component only need to be assembled and installed to contact with each other. The mounting bracket further stabilizes the contact structure between the two conducting paths. Compared with the pure wire connection available on the market, reliability in the present application is greatly improved.

The mounting bracket is inserted into the brush head handle in the electric toothbrush, so that the sonic motor body electromagnetically drives the hollow conductive motor shaft and thus the hollow conductive motor shaft drives the brush head handle to rotate reciprocally at a high-frequency through the mounting bracket. Finally, the brush head is driven to swing at a high frequency. The two electrical connectors are implemented as the conductive probe and conductive elastic plate respectively. The conductive probe is inserted into the first electrode. The conductive elastic plate is assembled with the mounting bracket via the guide slot. The structure of the mounting bracket achieves stable electrical contact, thereby achieving a high assembly performance and a good stability.

The power-consuming component is implemented as a lighting effect function component and can be connected to a power supply via the control main board. When the electric toothbrush is turned on, the lighting effect function component can be controlled by the control main board to operate, which can be applied according to a user's need.

The lighting effect function component can be implemented as multiple therapeutic equivalents, such as red light therapy, blue light whitening or violet light sterilization, etc. In combination with the brush head, a comprehensive effect of protecting gums and cleaning teeth is achieved.

The power-consuming component can be implemented as a sensor and/or a photographic component. The internal environment and conditions of the oral cavity can be visually known via the sensor and the photographic element. It can even provide a direct understanding of conditions such as gum inflammation and tooth decay.

### BRIEF DESCRIPTION OF THE DRAWINGS

FIG. 1 shows a schematic structural diagram of a conductive structure of a sonic motor in the present disclosure.

FIG. 2 shows an exploded structural diagram of a conductive structure of a sonic motor in the present disclosure.

FIG. 3 shows a schematic structural diagram of a first conductive plate in the present disclosure.

FIG. 4 shows a front view of a sonic motor body in the present disclosure.

FIG. 5 shows a sectional view along line A-A in FIG. 4.

FIG. 6 shows a schematic structural diagram of a brush head power supply structure for an electric toothbrush in the present disclosure.

FIG. 7 shows an exploded structural diagram of an mounting bracket and a hollow conductive motor shaft in FIG. 6.

FIG. 8 shows a schematic structural diagram of an electrical connection assembly in the present disclosure.

FIG. 9 shows a front view of a brush head handle in the present disclosure.

FIG. 10 shows a sectional view along line B-B in FIG. 9.

FIG. 11 shows a front view of an electric toothbrush in the present disclosure.

FIG. 12 shows a sectional view along C-C in FIG. 1.

FIG. 13 shows an enlarged view at portion D in FIG. 12.

### REFERENCE NUMBERS

| | |
|---|---|
| sonic motor body | 1 |
| hollow conductive motor shaft | 2 |
| output segment | 20 |
| first electrical output terminal | 201 |
| connection hole | 21 |
| motor shaft wire | 22 |
| first electrical input terminal | 221 |
| first conductive plate | 23 |
| flexible contact portion | 231 |
| first conductive component | 31 |
| insulating mounting base | 32 |
| second conductive component | 33 |
| second electrical input terminal | 331 |
| mounting bracket | 4 |
| guide slot | 40 |
| first mounting port | 41 |
| second mounting port | 42 |
| brush head | 5 |
| lighting effect function component | 51 |
| light board | 511 |
| light bead | 512 |
| brush head handle | 6 |
| insertion cavity | 60 |
| first electrical connector | 71 |
| elastic deformation portion | 711 |
| second electrical connector | 72 |
| control main board | 8 |
| battery | 9 |

### DETAILED DESCRIPTION

The preferred embodiments described below are only for illustration purposes. Those skilled in the art can conceive of other obvious variations. The basic principles of the present disclosure defined in the following description can be applied to other embodiments, variant technical solutions, improved technical solutions, equivalent technical solutions, and other technical solutions that do not deviate from the spirit and scope of the present disclosure.

Unless explicitly required by the context, similar words such as “include” and “comprise” in the entire specification and claims shall be interpreted as inclusive rather than exclusive or exhaustive. That is to say, it means “including but not limited to”. As for the words “and/or” contained in the text, they are used to simplify the expression. For example, “A and/or B” includes both “A and B” and “A or B”. The expression “A or B” is interpreted as “A” or “B”. The expression “A and B” are interpreted as the simultaneous selection of “A” and “B”.

An sonic electric motor or a sonic motor of an electric toothbrush, when supplied with an alternating forward and reverse current at a specific frequency, causes an output shaft of the motor to swing back and forth according to the frequency, so as to truly realize the action of “brushing teeth”. The vibration is caused by the attraction and repulsion between a stator and a rotor inside the motor. There is no mechanical friction inside the motor, so that the motor has a higher stability and a large output power, and thus can generate a high-frequency vibration and achieve an effect that is similar to “sound wave”. Based on above, the sonic motor is accepted and widely used by people in this field. The above content is necessary for understanding of the present application.

### Embodiment 1

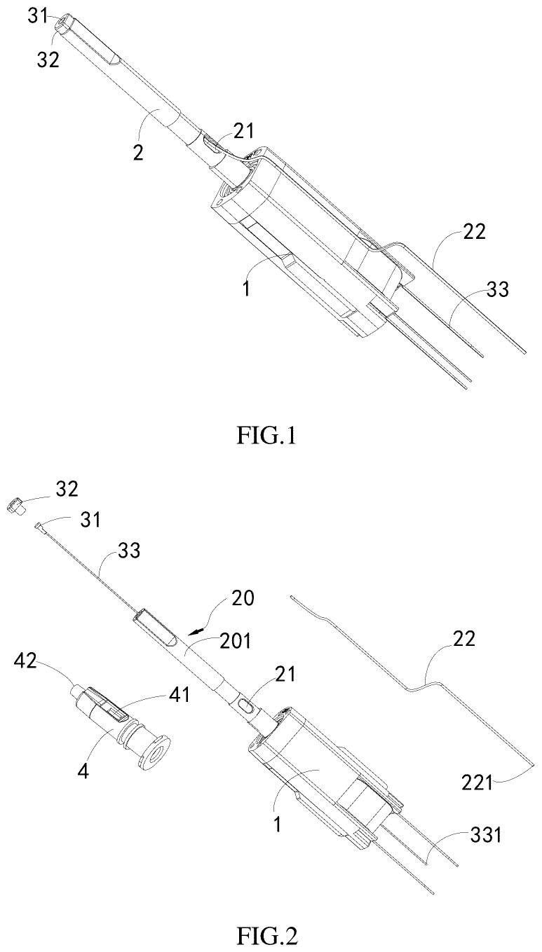

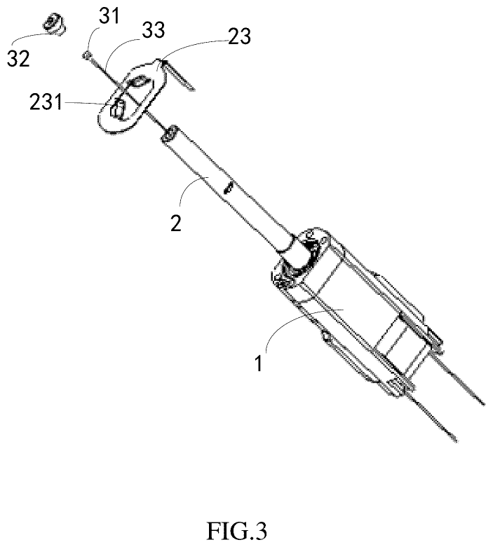

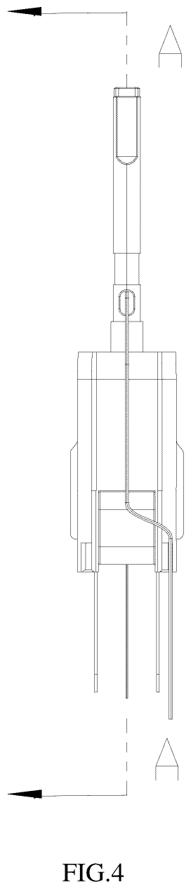

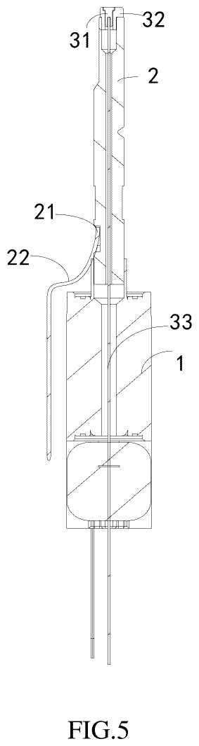

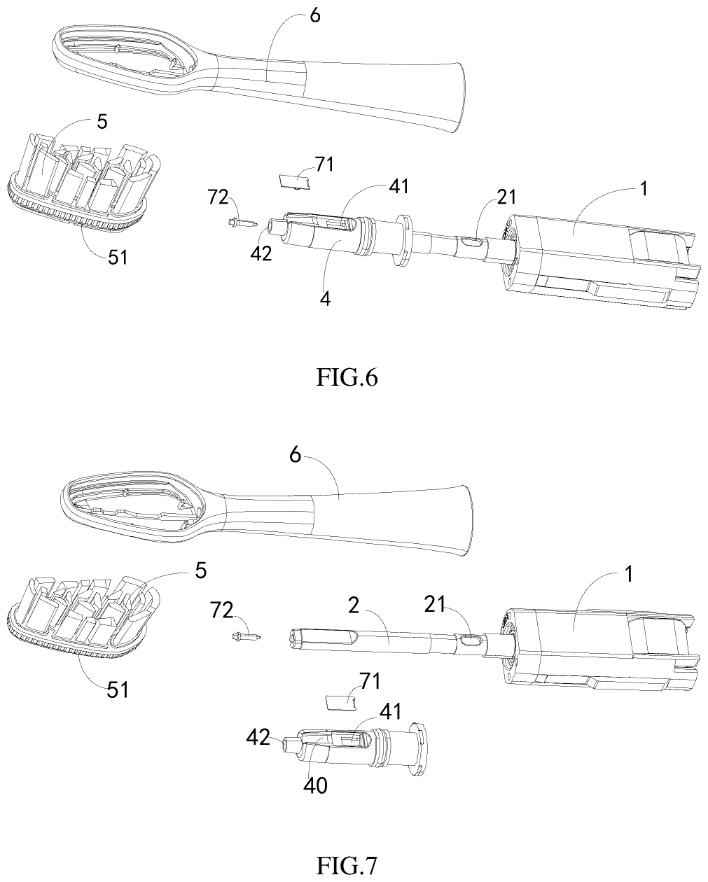

With reference to FIGS. 1 to 5 and in combination with FIGS. 6 and 7, in order to show a conducting path, a mounting bracket is not shown in FIG. 1 and FIG. 3, and the mounting bracket is entirely shown in FIG. 2, FIG. 6 and FIG. 7. First, the present disclosure provides a conductive structure of a sonic motor. The conductive structure includes a sonic motor body 1, a hollow conductive motor shaft 2 and a conductive assembly. One end of the hollow conductive motor shaft 2 passes through an interior of the sonic motor body 1, and is driven by the sonic motor body 1 to perform high-frequency reciprocating vibrations with a certain amplitude. A segment of the hollow conductive motor shaft 2 extends out from the sonic motor body 1 to form an output segment 20. The output segment 20 has a first electrical output terminal 201. The hollow conductive motor shaft 2 has a first electrical input terminal 221. The conductive assembly at least includes a first conductive component 31, a second conductive component 33 and an insulating mounting base 32. The first conductive component 31 is installed on the output segment 20 via the insulating mounting base 32 to form a second electrical output terminal. One end of the second conductive component 33 is connected to the first conductive component 31. Another end of the second conductive component 33 passes through the hollow conductive motor shaft 2 and extends out from the hollow conductive motor shaft 2 to form a second electrical input terminal 331.

As shown in FIG. 6 and FIG. 7, the mounting bracket 4 is sleeved on the hollow conductive motor shaft 2 and is at least circumferentially fixed relative to the hollow conductive motor shaft 2. The mounting bracket 4 has a first mounting port 41 at the first electrical output terminal 201 for mounting and connecting a power-consuming component. The mounting bracket 4 has a second mounting port 42 at the second electrical output terminal for mounting and connecting the power-consuming component. In the embodiment, the motor shaft of the sonic motor is implemented as a hollow conductive structure. An interior of the motor shaft is implemented as a wiring channel. The conductive assembly passes through and out of the body from the motor shaft along an axis of the motor shaft to connect to an element, so as to form a first conducting path. The motor shaft itself can be used as a second conducting path due to its conductive property. Both ends of each of the two conducting paths are connected to the power-consuming component and a power supply respectively, so as to form an electrically conductive path. The characteristics of the structure of the sonic motor itself is fully utilized. Only the motor shaft needs to be set to be hollow, and there is no need for a significant structural modification to the motor. Moreover, both the first electrical output terminal 201 and the second electrical output terminal are integrated with the motor shaft itself, thus the back-and-forth vibrations of the motor at a high frequency does not affect the electrical connection efficiency and no extra wiring layout is required. The two output terminals can be mounted to an external part in an assembled way and thus have incomparable advantages in both security and convenience.

The above contents are necessary for implementing this embodiment. The following is a further detailed description in combination with the attached drawings.

In the embodiment, the sonic motor body 1 is implemented as a structure of the sonic motor except for the motor shaft, and includes a casing, a stator, a rotor and bearing parts in the openings at both ends of the casing. This is a principle structure of the sonic electric motor and the sonic motor. This embodiment does not make technical improvements on the principle structure, and thus details of the principle structure are omitted here.

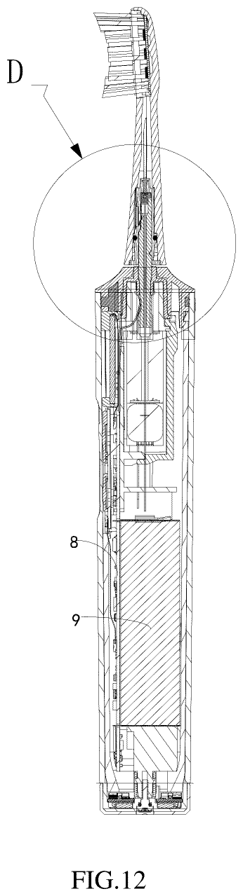

As a preferred embodiment of the present disclosure, with reference to FIG. 1 and FIG. 2, the hollow conductive motor shaft 2 further includes a motor shaft wire 22. One end of the motor shaft wire 22 is connected to the hollow conductive motor shaft 2 so as to be in electrical connection with the first electrical output terminal 201, and another end of the motor shaft wire 22 is implemented as the first electrical input terminal 221. In the embodiment, the other end of the motor shaft wire 22 is connected with a control main board 8 or a battery 9 (as shown in FIG. 12) of the power supply component. The power-consuming component consumes power through connecting to the first electrical output terminal 201. Preferably, the motor shaft wire 22 is wrapped with insulating plastic to prevent electric leakage. When in the process of arranging the motor shaft wire, a maximum amplitude of a reciprocating swing of the hollow conductive motor shaft 2 should be taken into account and the reserved space length of the motor shaft wire 22 should ensure that the motor shaft wire 22 will not break off.

Furthermore, the motor shaft wire 22 can be arranged either inside or outside the hollow conductive motor shaft 2. When the motor shaft wire 22 is arranged outside the hollow conductive motor shaft 2, as shown in FIG. 1, a connection hole 21 is formed on a side wall of the output segment 20 of the hollow conductive motor shaft 2. One end of the motor shaft wire 22 is connected to the hollow conductive motor shaft 2 in a hidden way via the connection hole 21. The other end of the motor shaft wire 22 extends outside the hollow conductive motor shaft 2 and extends to the power supply component.

Alternatively, when the motor shaft wire 22 is arranged inside the hollow conductive motor shaft 2, a connection hole 21 is formed on a side wall of the output segment 20 of the hollow conductive motor shaft 2. One end of the motor shaft wire 22 is connected to the hollow conductive motor shaft 2 in a hidden way via the connection hole 21. The other end of the motor shaft wire 22 extends within the hollow conductive motor shaft 2.

Furthermore, as shown in FIG. 3, in the embodiment, a first conductive plate 23 is included. The first conductive plate 23 is mounted on the sonic motor body 1 and is in contact with the hollow conductive motor shaft 2. The one end of the motor shaft wire 22 is connected to the first conductive plate 23, which is convenient for an electrical conducting between the motor shaft wire 22 and the hollow conductive motor shaft 2. Preferably, there is a through hole in the middle of the first conductive plate 23. and there is a flexible contact portion 231 in the through hole. The hollow conductive motor shaft 2 passes through the through hole. The flexible contact portion 231 is pressed against the hollow conductive motor shaft 2 for electrical contact. The first conductive plate 23 can be installed on the sonic motor body 1. Preferably, there are two flexible contact portions 231 and the two flexible contact portions 231 are located on the two sides of the hollow conductive motor shaft 2 and is in contact with the hollow conductive motor shaft 2. The flexible contact portion 231 can be implemented as a metal wire or a strand of metal wires, etc.

It should be noted that in this embodiment, the hollow conductive motor shaft 2 is used as a conductive bridge for the electrical connection between the power-consuming component and the power supply component. A connection form between the power-consuming component and the hollow conductive motor shaft 2 and a connection form between the power supply component and the hollow conductive motor shaft 2 should not be limited. The contact connection using the first conductive plate 23, and the connection using the motor shaft wire 22 are described above, and the connection can be achieved by any other way. The connection form is not limited to any of the above examples. The first electrical input terminal 221 and the second electrical input terminal 331 are connected to the power supply component by directly using a positive wire and a negative wire or by a contact connection using e.g., an electrode sheet, a metal resilient plate that can be mounted. The particular connection manner is selected based on implementation criteria.

Furthermore, the first conductive component 31 is a first electrode, and the second conductive component 33 is a second wire. The first electrode is installed at a rear end of the output segment 20 via the insulating mounting base 32. The second wire passes through the hollow conductive motor shaft 2 and is connected to the first electrode. The second electrical input terminal 331 is defined as an end of the second wire that is far away from the first electrode. The second electrical output terminal is defined as the first electrode.

Furthermore, with reference to FIGS. 6, 7 and 8, in order to facilitate the connection with power-consuming component, an electrical connection assembly is included in this embodiment. The electrical connection assembly includes a first electrical connector 71 and a second electrical connector 72. The first electrical connector 71 is detachably mounted at the first mounting port 41 and is in contact with the first electrical output terminal 201. The second electrical connector 72 is detachably mounted at the second mounting port 42 and is in contact with the second electrical output terminal. The first electrical connector 71 and the second electrical connector 72 are connected to positive and negative electrodes of the power-consuming component through wires to provide power.

As a preferred embodiment of the present disclosure, the first electrical connector 71 is implemented as conductive elastic plate. An elastic deformation portion 711 is provided at a bottom of the conductive elastic plate. A guide slot 40 is provided on the mounting bracket 4 along an insertion direction. The first mounting port 41 is located in the guide slot 40. The conductive elastic plate compresses the elastic deformation portion 711 so as to be inserted along the guide slot 40 into the first mounting port 41. The elastic deformation portion 711 extends into the first mounting port 41 and is in electrical contact with the first electrical output terminal 201.

Furthermore, the second electrical connector 72 is a conductive probe. The conductive probe is in electrical contact with the first electrode. The power-consuming component can be connected to the conductive probe and the conductive elastic plate only through wires. Preferably, the conductive elastic plate is implemented as a deformable flat spring, and the deformable portion corresponds to the elastic deformation portion 711.

The power supply component in the embodiment can be implemented as a control main board 8. The power-consuming component is implemented as a lighting effect function component, a sensor or a photographic component arranged in the brush head 5 of the electric toothbrush.

Please refer to FIG. 2, a conductive path forming a conducting circuit in this embodiment is as follows.

Firstly, the first electrical input terminal 221 and the second electrical input terminal 331 are respectively connected to a positive electrode and a negative electrode of the power supply component. Then, the first electrical input terminal 221 is connected to the connection hole 21 of the output segment 20 via the motor shaft wire 22, and the first electrical output terminal 201 on the output segment 20 of the motor shaft conducts electricity, so as to form a first path. The second electrical input terminal 331 is connected to the first conductive component 31 (implemented as the first electrode and defined as the second electrical output terminal) via the second conductive component 33 (implemented as the second wire) to form a second path. The insulating property of the insulating mounting base 32 and the second wire prevent a short circuit between the two paths. Finally, the positive and negative terminals of the power-consuming component are connected to the first electrical output terminal 201 and the first electrode.

Beneficial effects of the above technical solutions may result from any one of the following items or any combination thereof.

In the present disclosure, the motor shaft of the sonic motor is implemented as a hollow conductive structure. An interior of the motor shaft is implemented as a wiring channel. The conductive assembly passes through and out of the body from the motor shaft along an axis of the motor shaft for connecting to an element, so as to form a first conducting path. The motor shaft itself can be used as a second conducting path due to its conductive property. Both ends of each of the two conducting paths are connected to the power-consuming component and a power supply respectively, so as to form an electrically conductive path. The characteristics of the structure of the sonic motor itself is fully utilized. Only the motor shaft needs to be set to be hollow, and there is no need for a significant structural modification to the motor. Moreover, both the first electrical output terminal 201 and the second electrical output terminal are integrated with the motor shaft itself, thus the back-and-forth vibrations of the motor at a high frequency does not affect the electrical connection efficiency and no extra wiring layout is required. The two output terminals can be contact with corresponding inner parts stably via the mounting bracket 4 and can be mounted with an external part in an assembled way, and thus has incomparable advantages in both security and convenience.

The external output linkage is completed through the mounting bracket 4. At the same time, due to a structure of the mounting bracket 4, the power-consuming component can be installed on the mounting bracket 4 and can be assembled with the mounting ports on the mounting bracket 4, so that electrical connection of the two output terminals can be achieved.

Wire paths can be completely routed inside the hollow conductive motor shaft 2, or one wire path can be led out from the hollow conductive motor shaft 2 via the motor shaft wire 22 to connect to an external element. In this way, the present application has a strong adaptability and a wide range of application. It is particularly suitable for a conductive structure in which the power supply is located on one side of the sonic motor body 1 and the power-consuming component is located on the other side of the sonic motor body 1. It has a strong integration, a high space utilization and low difficulty of arranging wires, and improves the durability.

The two electrical output terminals are connected through the electrical connection assembly in an assembled way, and the assembling is very convenient. The conducting paths are clear. The conducting path of the power supply structure is integrated on the sonic motor, and the conducting path of the power-consuming component is integrated into the structure of the power-consuming component. The conducting path of the power supply structure and the conducting path of the power-consuming component only need to be assembled and installed to contact with each other. The mounting bracket 4 further stabilizes the contact structure between the two conducting paths. Compared with the pure wire connection available on the market, reliability in the present application is greatly improved.

### Embodiment 2

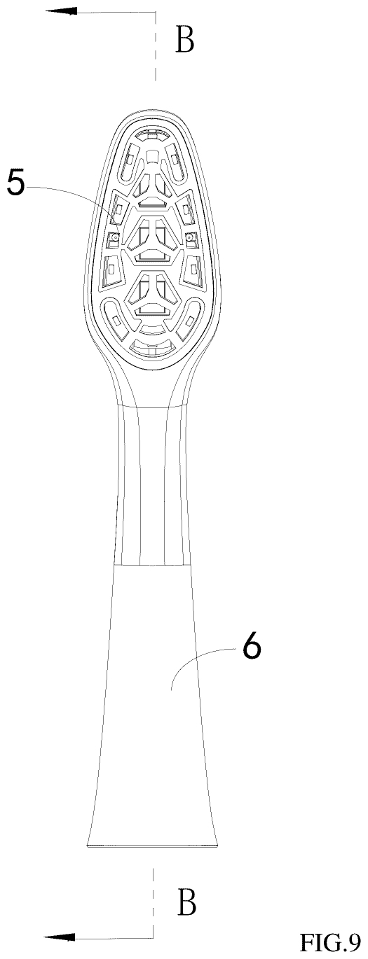

With reference to FIGS. 6 to 10 and in combination with FIGS. 1 to 5, the present embodiment provides a brush head power supply structure for an electric toothbrush. The brush head power supply structure at least includes a brush head 5 and a conductive structure of a sonic motor. A power-consuming component that needs to consume power is arranged in the brush head 5. A positive electrode and a negative electrode of the power-consuming component are correspondingly connected to the first electrical output terminal 201 and the second electrical output terminal.

Furthermore, the conductive structure of the sonic motor includes a sonic motor body 1, a hollow conductive motor shaft 2 and a conductive assembly. One end of the hollow conductive motor shaft 2 passes through an interior of the sonic motor body 1, and is driven by the sonic motor body 1 to perform high-frequency reciprocating vibrations with a certain amplitude. A segment of the hollow conductive motor shaft 2 extends out from the sonic motor body 1 to form an output segment 20. The output segment 20 has a first electrical output terminal 201. The hollow conductive motor shaft 2 has a first electrical input terminal 221. The conductive assembly at least includes a first conductive component 31, a second conductive component 33 and an insulating mounting base 32. The first conductive component 31 is installed on the output segment 20 via the insulating mounting base 32 to form a second electrical output terminal. One end of the second conductive component 33 is connected to the first conductive component 31. Another end of the second conductive component 33 passes through the hollow conductive motor shaft 2 and extends out from the hollow conductive motor shaft 2 to form a second electrical input terminal 331.

As shown in FIG. 6 and FIG. 7, the mounting bracket 4 is sleeved on the hollow conductive motor shaft 2 and is at least circumferentially fixed relative to the hollow conductive motor shaft 2. The mounting bracket 4 has a first mounting port 41 at the first electrical output terminal 201 for mounting and connecting a power-consuming component. The mounting bracket 4 has a second mounting port 42 at the second electrical output terminal 42 for mounting and connecting the power-consuming component.

In the embodiment, the sonic motor body 1 is implemented as a structure of the sonic motor except for the motor shaft, and includes a casing, a stator, a rotor and bearing parts in the openings at both ends of the casing. This is a principle structure of the sonic electric motor and the sonic motor. This embodiment does not make technical improvements on the principle structure, and thus details of the principle structure are omitted here.

As a preferred embodiment of the present disclosure, with reference to FIG. 1 and FIG. 2, the hollow conductive motor shaft 2 further includes a motor shaft wire 22. One end of the motor shaft wire 22 is connected to the hollow conductive motor shaft 2 so as to be in electrical connection with the first electrical output terminal 201, and another end of the motor shaft wire 22 is implemented as the first electrical input terminal 221. In the embodiment, the other end of the motor shaft wire 22 is connected with a control main board 8 or a battery 9 (as shown in FIG. 12) of the power supply component. The power-consuming component consumes power through connecting to the first electrical output terminal 201. Preferably, the motor shaft wire 22 is wrapped with insulating plastic to prevent electric leakage. When in the process of arranging the motor shaft wire, a maximum amplitude of a reciprocating swing of the hollow conductive motor shaft 2 should be taken into account and the reserved space length of the motor shaft wire 22 should ensure that the motor shaft wire 22 will not break off.

Furthermore, the motor shaft wire 22 can be arranged either inside or outside the hollow conductive motor shaft 2. When the motor shaft wire 22 is arranged outside the hollow conductive motor shaft 2, as shown in FIG. 1, a connection hole 21 is formed on a side wall of the output segment 20 of the hollow conductive motor shaft 2. One end of the motor shaft wire 22 is connected to the hollow conductive motor shaft 2 in a hidden way via the connection hole 21. The other end of the motor shaft wire 22 extends outside the hollow conductive motor shaft 2 and extends to the power supply component.

Alternatively, when the motor shaft wire 22 is arranged inside the hollow conductive motor shaft 2, a connection hole 21 is formed on a side wall of the output segment 20 of the hollow conductive motor shaft 2. One end of the motor shaft wire 22 is connected to the hollow conductive motor shaft 2 in a hidden way via the connection hole 21. The other end of the motor shaft wire 22 extends within the hollow conductive motor shaft 2.

Furthermore, as shown in FIG. 3, in the embodiment, a first conductive plate 23 is included. The first conductive plate 23 is mounted on the sonic motor body 1 and is in contact with the hollow conductive motor shaft 2. The one end of the motor shaft wire 22 is connected to the first conductive plate 23, which is convenient for an electrical conducting between the motor shaft wire 22 and the hollow conductive motor shaft 2. Preferably, there is a through hole in the middle of the first conductive plate 23. and there is a flexible contact portion 231 in the through hole. The hollow conductive motor shaft 2 passes through the through hole. The flexible contact portion 231 is pressed against the hollow conductive motor shaft 2 for electrical contact. The first conductive plate 23 can be installed on the sonic motor body 1. Preferably, there are two flexible contact portions 231 and the two flexible contact portions 231 are located on the two sides of the hollow conductive motor shaft 2 and is in contact with the hollow conductive motor shaft 2. The flexible contact portion 231 can be implemented as a metal wire or a strand of metal wires, etc.

Furthermore, the first conductive component 31 is a first electrode, and the second conductive component 33 is a second wire. The first electrode is installed at a rear end of the output segment 20 via the insulating mounting base 32. The second wire passes through the hollow conductive motor shaft 2 and is connected to the first electrode. The second electrical input terminal 331 is defined as an end of the second wire that is far away from the first electrode. The second electrical output terminal is defined as the first electrode.

With reference to FIGS. 6, 7 and 8, in the present embodiment, a brush head handle 6 is further included. The brush head 5 is arranged on the brush head handle 6, and is in indirect transmission with the sonic motor via the brush head handle 6. The brush head handle 6 has an insertion cavity 60 and an electrical connection assembly is provided in the insertion cavity 60. The electrical connection assembly includes a first electrical connector 71 and a second electrical connector 72. The first electrical connector 71 and the second electrical connector 72 are respectively connected to a positive electrode and a negative electrode of the power-consuming component through wires. The mounting bracket 4 is inserted into the insertion cavity 60 of the brush head handle 6. The first electrical connector 71 is detachably mounted at the first mounting port 41 and is in contact with the first electrical output terminal 201, the second electrical connector 72 is detachably mounted at the second mounting port 42 and is in contact with the second electrical output terminal. The first electrical connector 71 and the second electrical connector 72 are connected to positive and negative electrodes of the power-consuming component through wires to provide power.

As a preferred embodiment of the present disclosure, the first electrical connector 71 is implemented as conductive elastic plate. An elastic deformation portion 711 is provided at a bottom of the conductive elastic plate. A guide slot 40 is provided on the mounting bracket 4 along an insertion direction. The first mounting port 41 is located in the guide slot 40. The conductive elastic plate compresses the elastic deformation portion 711 so as to be inserted along the guide slot 40 into the first mounting port 41. The elastic deformation portion 711 extends into the first mounting port 41 and is in electrical contact with the first electrical output terminal 201.

Furthermore, the second electrical connector 72 is a conductive probe. The conductive probe is in electrical contact with the first electrode. The power-consuming component can be connected to the conductive probe and the conductive elastic plate only through wires. Preferably, the conductive elastic plate is implemented as a deformable flat spring, and the deformable portion corresponds to the elastic deformation portion 711.

The power supply component in the embodiment can be implemented as a control main board 8. The power-consuming component is implemented as a lighting effect function component, a sensor or a photographic component arranged in the brush head 5 of the electric toothbrush.

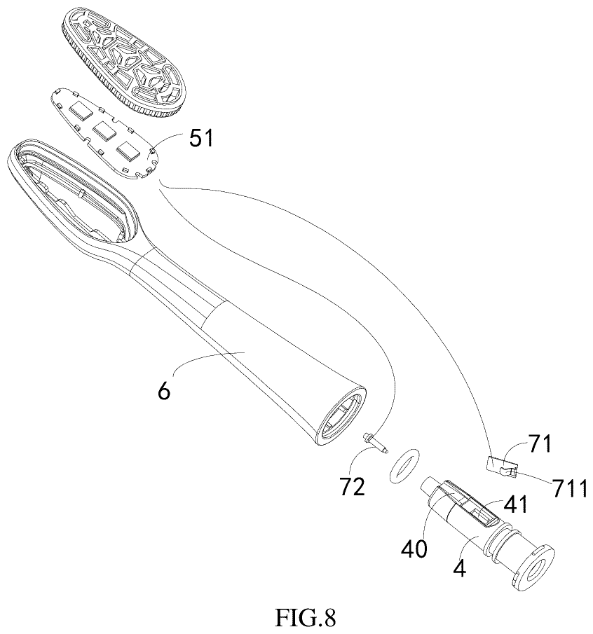

Please refer to FIG. 2 and in combination with FIGS. 7 and 8, the conductive path forming circuit in this embodiment is that:

Firstly, the first electrical input terminal 221 and the second electrical input terminal 331 are respectively connected to a positive electrode and a negative electrode of the power supply component. Then, the first electrical input terminal 221 is connected to the connection hole 21 of the output segment 20 via the motor shaft wire 22, and the first electrical output terminal 201 on the output segment 20 of the motor shaft conducts electricity so as to form a first path. The second electrical input terminal 331 is connected to the first conductive component 31 (implemented as the first electrode and defined as the second electrical output terminal) via the second conductive component 33 (implemented as the second wire) to form a second path. The insulating property of the insulating mounting base 32 and the second wire prevent a short circuit between the two paths. Finally, the brush head 5 is inserted into the mounting bracket 4 via the brush head handle 6. The lighting effect function component 51 is connected to the conductive elastic plate and the conductive probe through wires. With the installation of the brush head handle 6, the conductive elastic plate is inserted along the guide slot 40 to the first mounting port 41, and then the elastic deformation portion 711 pops out and contacts the first electrical output terminal 201. In addition, with the installation of the brush head handle 6, the conductive probe is inserted into the second mounting port 42 and contacts with the first electrode (that is, the second electrical output terminal) to form a path.

The present disclosure has the following beneficial effects.

In the present disclosure, the motor shaft of the sonic motor is implemented as a hollow conductive structure. An interior of the motor shaft is implemented as a wiring channel. The conductive assembly passes through and out of the body from the motor shaft along an axis of the motor shaft for connecting to an element, so as to form a first conducting path. The motor shaft itself can be used as a second conducting path due to its conductive property. Both ends of each of the two conducting paths are connected to the power-consuming component and a power supply respectively, so as to form an electrically conductive path. The characteristics of the structure of the sonic motor itself is fully utilized. Only the motor shaft needs to be set to be hollow, and there is no need for a significant structural modification to the motor. Moreover, both the first electrical output terminal 201 and the second electrical output terminal are integrated with the motor shaft itself, thus the back-and-forth vibrations of the motor at a high frequency does not affect the electrical connection efficiency and no extra wiring layout is required. The two output terminals can be contact with corresponding inner parts stably via the mounting bracket 4 and can be mounted with an external part in an assembled way and thus has incomparable advantages in both security and convenience.

The external output linkage is completed through the mounting bracket 4. At the same time, due to a structure of the mounting bracket 4, the power-consuming component can be installed on the mounting bracket 4 and can be assembled with the mounting ports on the mounting bracket 4, so that electrical connection of the two output terminals can be achieved.

Wire paths can be completely routed inside the hollow conductive motor shaft 2, or one wire path can be led out from the hollow conductive motor shaft 2 via the motor shaft wire 22 to connect to an external element. In this way, the present application has a strong adaptability and a wide range of application. It is particularly suitable for a conductive structure in which the power supply is located on one side of the sonic motor body 1 and the power-consuming component is located on the other side of the sonic motor body 1. It has a strong integration, a high space utilization and low difficulty of arranging wires, and improves the durability.

The two electrical output terminals are connected through the electrical connection assembly in an assembled way, and the assembling is very convenient. The conducting paths are clear. The conducting path of the power supply structure is integrated on the sonic motor, and the conducting path of the power-consuming component is integrated into the structure of the power-consuming component. The conducting path of the power supply structure and the conducting path of the power-consuming component only need to be assembled and installed to contact with each other. The mounting bracket 4 further stabilizes the contact structure between two conducting paths. Compared with the pure wire connection available on the market, reliability in the present application is greatly improved.

The mounting bracket 4 is inserted into the brush head handle 6 in the electric toothbrush, so that the sonic motor body 1 electromagnetically drives the hollow conductive motor shaft 2 and thus the hollow conductive motor shaft 2 drives the brush head handle 6 to rotate reciprocally at a high-frequency through the mounting bracket 4. Finally, the brush head 5 is driven to swing at a high frequency. The two electrical connectors are implemented as the conductive probe and conductive elastic plate respectively. The conductive probe is inserted into the first electrode. The conductive elastic plate is assembled with the mounting bracket 4 via the guide slot. The structure of the mounting bracket 4 achieves stable electrical contact, thereby achieving a high assembly performance and a good stability.

### Embodiment 3

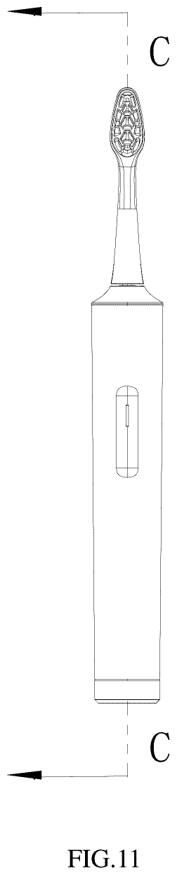

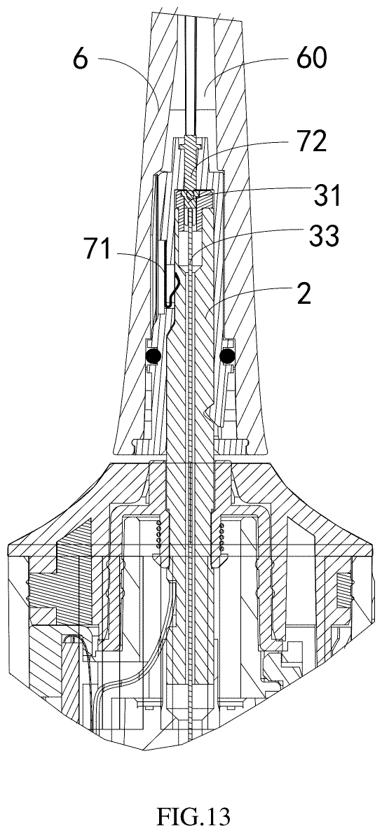

With reference to FIGS. 11 to 13, the present disclosure provides an electric toothbrush which includes the brush head power supply structure for an electric toothbrush. The first electrical input terminal 221 and the second electrical input terminal 331 are connected to a control main board 8 or a battery 9 of the electric toothbrush.

It should be noted that the electric toothbrush includes a main body, a brush head 5, a brush head handle 6, and other structures. The principle thereof belongs to the common technical knowledge in this field, so there is no principle introduction. In the embodiment, the electric toothbrush includes the main body and the brush head power supply structure for an electric toothbrush. Furthermore, the brush head power supply structure includes the conductive structure of the sonic motor, the brush head 5 and the brush head handle 6.

Specifically, with reference to FIGS. 6 to 10 and in combination with FIGS. 1 to 5, the conductive structure of the sonic motor includes a sonic motor body 1, a hollow conductive motor shaft 2 and a conductive assembly. One end of the hollow conductive motor shaft 2 passes through an interior of the sonic motor body 1, and is driven by the sonic motor body 1 to perform high-frequency reciprocating vibrations with a certain amplitude. A segment of the hollow conductive motor shaft 2 extends out from the sonic motor body 1 to form an output segment 20. The output segment 20 has a first electrical output terminal 201. The hollow conductive motor shaft 2 has a first electrical input terminal 221. The conductive assembly at least includes a first conductive component 31, a second conductive component 33 and an insulating mounting base 32. The first conductive component 31 is installed on the output segment 20 via the insulating mounting base 32 to form a second electrical output terminal. One end of the second conductive component 33 is connected to the first conductive component 31. Another end of the second conductive component 33 passes through the hollow conductive motor shaft 2 and extends out from the hollow conductive motor shaft 2 to form a second electrical input terminal 331. As shown in FIGS. 6 and 7, the mounting bracket 4 is sleeved on the hollow conductive motor shaft 2 and is at least circumferentially fixed relative to the hollow conductive motor shaft 2. The mounting bracket 4 has a first mounting port 41 at the first electrical output terminal 201 for mounting and connecting a power-consuming component. The mounting bracket 4 has a second mounting port 42 at the second electrical output terminal 42 for mounting and connecting the power-consuming component.

In the embodiment, the sonic motor body 1 is implemented as a structure of the sonic motor except for the motor shaft, and includes a casing, a stator, a rotor and bearing parts in the openings at both ends of the casing. This is a principle structure of the sonic electric motor and the sonic motor. This embodiment does not make technical improvements on the principle structure, and thus details of the principle structure are omitted here.

As a preferred embodiment of the present disclosure, with reference to FIG. 1 and FIG. 2, the hollow conductive motor shaft 2 further includes a motor shaft wire 22. One end of the motor shaft wire 22 is connected to the hollow conductive motor shaft 2 so as to be in electrical connection with the first electrical output terminal 201, and another end of the motor shaft wire 22 is implemented as the first electrical input terminal 221. In the embodiment, the other end of the motor shaft wire 22 is connected with a control main board 8 or a battery 9 (as shown in FIG. 12) of the power supply component. The power-consuming component consumes power through connecting to the first electrical output terminal 201. Preferably, the motor shaft wire 22 is wrapped with insulating plastic to prevent electric leakage. When in the process of arranging the motor shaft wire, a maximum amplitude of a reciprocating swing of the hollow conductive motor shaft 2 should be taken into account and the reserved space length of the motor shaft wire 22 should ensure that the motor shaft wire 22 will not break off.

Furthermore, the motor shaft wire 22 can be arranged either inside or outside the hollow conductive motor shaft 2. When the motor shaft wire 22 is arranged outside the hollow conductive motor shaft 2, as shown in FIG. 1, a connection hole 21 is formed on a side wall of the output segment 20 of the hollow conductive motor shaft 2. One end of the motor shaft wire 22 is connected to the hollow conductive motor shaft 2 in a hidden way via the connection hole 21. The other end of the motor shaft wire 22 extends outside the hollow conductive motor shaft 2 and extends to the power supply component.

Alternatively, when the motor shaft wire 22 is arranged inside the hollow conductive motor shaft 2, a connection hole 21 is formed on a side wall of the output segment 20 of the hollow conductive motor shaft 2. One end of the motor shaft wire 22 is connected to the hollow conductive motor shaft 2 in a hidden way via the connection hole 21. The other end of the motor shaft wire 22 extends within the hollow conductive motor shaft 2.

Furthermore, as shown in FIG. 3, in the embodiment, a first conductive plate 23 is included. The first conductive plate 23 is mounted on the sonic motor body 1 and is in contact with the hollow conductive motor shaft 2. The one end of the motor shaft wire 22 is connected to the first conductive plate 23, which is convenient for an electrical conducting between the motor shaft wire 22 and the hollow conductive motor shaft 2. Preferably, there is a through hole in the middle of the first conductive plate 23. and there is a flexible contact portion 231 in the through hole. The hollow conductive motor shaft 2 passes through the through hole. The flexible contact portion 231 is pressed against the hollow conductive motor shaft 2 for electrical contact. The first conductive plate 23 can be installed on the sonic motor body 1. Preferably, there are two flexible contact portions 231 and the two flexible contact portions 231 are located on the two sides of the hollow conductive motor shaft 2 and is in contact with the hollow conductive motor shaft 2. The flexible contact portion 231 can be implemented as a metal wire or a strand of metal wires, etc.

Furthermore, the first conductive component 31 is a first electrode, and the second conductive component 33 is a second wire. The first electrode is installed at a rear end of the output segment 20 via the insulating mounting base 32. The second wire passes through the hollow conductive motor shaft 2 and is connected to the first electrode. The second electrical input terminal 331 is defined as an end of the second wire that is far away from the first electrode. The second electrical output terminal is defined as the first electrode.

With reference to FIGS. 6, 7 and 8, in the present embodiment, a brush head handle 6 is further included. The brush head 5 is arranged on the brush head handle 6, and is in indirect transmission with the sonic motor via the brush head handle 6. The brush head handle 6 has an insertion cavity 60 and an electrical connection assembly is provided in the insertion cavity 60. The electrical connection assembly includes a first electrical connector 71 and a second electrical connector 72. The first electrical connector 71 and the second electrical connector 72 are respectively connected to a positive electrode and a negative electrode of the power-consuming component through wires. The mounting bracket 4 is inserted into the insertion cavity 60 of the brush head handle 6. The first electrical connector 71 is detachably mounted at the first mounting port 41 and is in contact with the first electrical output terminal 201, the second electrical connector 72 is detachably mounted at the second mounting port 42 and is in contact with the second electrical output terminal. The first electrical connector 71 and the second electrical connector 72 are connected to positive and negative electrodes of the power-consuming component through wires to provide power.

As a preferred embodiment of the present disclosure, the first electrical connector 71 is implemented as conductive elastic plate. An elastic deformation portion 711 is provided at a bottom of the conductive elastic plate. A guide slot 40 is provided on the mounting bracket 4 along an insertion direction. The first mounting port 41 is located in the guide slot 40. The conductive elastic plate compresses the elastic deformation portion 711 to allow the elastic deformation portion to be inserted along the guide slot 40 into the first mounting port 41. The elastic deformation portion 711 extends into the first mounting port 41 and is in electrical contact with the first electrical output terminal 201.

Furthermore, the second electrical connector 72 is a conductive probe. The conductive probe is in electrical contact with the first electrode. The power-consuming component can be connected to the conductive probe and the conductive elastic plate only through wires. Preferably, the conductive elastic plate is implemented as a deformable flat spring, and the deformable portion corresponds to the elastic deformation portion 711.

The power supply component in the embodiment can be implemented as a control main board 8 and a battery. The power-consuming component is implemented as a lighting effect function component, a sensor or a photographic component arranged in the brush head 5 of the electric toothbrush.

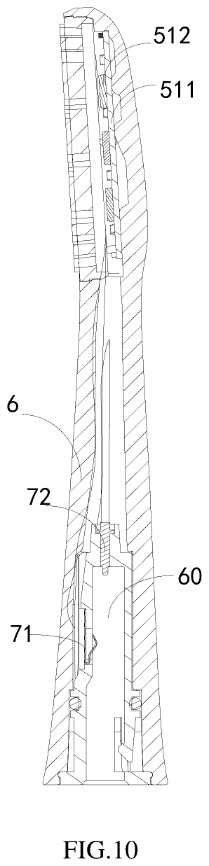

With reference to FIG. 10, in the embodiment, the power-consuming component is a lighting effect function component 51. The lighting effect function component 51 includes a light board 511 and a light bead 512. The light board 511 is installed in the brush head 5, the light board 511 is electrically connected to the first electrical output terminal 201 and the second electrical output terminal. The light bead 512 is installed on the light board 511. Furthermore, the light bead 512 is implemented as one of a red light therapy bead, a blue light brightening bead or a violet light sterilization bead or any combination thereof.

In order to facilitate the better action of the therapeutic light of the light bead 512 on the mouth, bristles of the electric toothbrush are at least partially implemented as light-conducting bristles. Preferably, the location of the light-conducting bristles correspond to the location of the light bead 512 so that the light from the light bead 512 is easily exported along the light-conducting bristles. Specifically, The light-conducting bristles are transparent bristles or bristles into which a light guide agent has been added during preparation.

Furthermore, the power-consuming component is implemented as a data acquisition device such as a sensor and/or a photographic component, so as to collect environmental data from the mouth, etc.

With reference to FIG. 2 and in combination with FIGS. 7, 8, 12 and 13, the conductive path forming circuit in this embodiment is as follows.

Firstly, the first electrical input terminal 221, the second electrical input terminal 331 and the control main board 8 are connected to a positive electrode and a negative electrode of the battery 9. Then the first electrical input terminal 221 is connected to the connection hole 21 of the output segment 20 via the motor shaft wire 22. At the same time, the first electrical output terminal 201 on the output segment 20 of the motor shaft conducts electricity to form a first path. The second electrical input terminal 331 is connected to the first conductive component 31 (implemented as the first electrode and defined as the second electrical output terminal) via the second conductive component 33 (implemented as the second wire) to form a second path. The insulation characteristic of the insulating mounting base 32 and the second wire prevent a short circuit between the two paths. Finally, the brush head 5 is installed by inserting the mounting bracket 4 into the brush head handle 6. The lighting effect function component 51 is connected with the conductive elastic plate and the conductive probe through wires. With the installation of the brush head handle 6, the conductive elastic plate is inserted along the guide slot 40 to the first mounting port 41, and then the elastic deformation portion 711 pops out and contacts the first electrical output terminal 201. In addition, with the installation of the brush head handle 6, the conductive probe is inserted into the second mounting port 42 and contacts with the first electrode (that is, the second electrical output terminal) to form a path.

The present disclosure has the following beneficial effects.

In the present disclosure, the motor shaft of the sonic motor is implemented as a hollow conductive structure. An interior of the motor shaft is implemented as a wiring channel. The conductive assembly passes through and out of the body from the motor shaft along an axis of the motor shaft for connecting to an element, so as to form a first conducting path. The motor shaft itself can be used as a second conducting path due to its conductive property. Both ends of each of the two conducting paths are connected to the power-consuming component and a power supply respectively, so as to form an electrically conductive path. The characteristics of the structure of the sonic motor itself is fully utilized. Only the motor shaft needs to be set to be hollow, and there is no need for a significant structural modification to the motor. Moreover, both the first electrical output terminal 201 and the second electrical output terminal are integrated with the motor shaft itself, thus the back-and-forth vibrations of the motor at a high frequency does not affect the electrical connection efficiency and no extra wiring layout is required. The two output terminals can be contact with corresponding inner parts stably via the mounting bracket 4 and can be mounted with an external part in an assembled way, and thus has incomparable advantages in both security and convenience.

The external output linkage is completed through the mounting bracket 4. At the same time, due to a structure of the mounting bracket 4, the power-consuming component can be installed on the mounting bracket 4 and can be assembled with the mounting ports on the mounting bracket 4, so that electrical connection of the two output terminals can be achieved.

Wire paths can be completely routed inside the hollow conductive motor shaft 2, or one wire path can be led out from the hollow conductive motor shaft 2 via the motor shaft wire 22 to connect to an external element. In this way, the present application has a strong adaptability and a wide range of application. It is particularly suitable for a conductive structure in which the power supply is located on one side of the sonic motor body 1 and the power-consuming component is located on the other side of the sonic motor body 1. It has a strong integration, a high space utilization and low difficulty of arranging wires, and improves the durability.

The two electrical output terminals are connected through the electrical connection assembly in an assembled way, and the assembling is very convenient. The conducting paths are clear. The conducting path of the power supply structure is integrated on the sonic motor, and the conducting path of the power-consuming component is integrated into the structure of the power-consuming component. The conducting path of the power supply structure and the conducting path of the power-consuming component only need to be assembled and installed to contact with each other. The mounting bracket 4 further stabilizes the contact structure between the two conducting paths. Compared with the pure wire connection available on the market, reliability in the present application is greatly improved.

The mounting bracket 4 is inserted into the brush head handle 6 in the electric toothbrush, so that the sonic motor body 1 electromagnetically drives the hollow conductive motor shaft 2 and thus the hollow conductive motor shaft 2 drives the brush head handle 6 to rotate reciprocally at a high-frequency through the mounting bracket 4. Finally, the brush head 5 is driven to swing at a high frequency. The two electrical connectors are implemented as the conductive probe and conductive elastic plate respectively. The conductive probe is inserted into the first electrode. The conductive elastic plate is assembled with the mounting bracket 4 via the guide slot. The structure of the mounting bracket 4 achieves stable electrical contact, thereby achieving a high assembly performance and a good stability.

The power-consuming component is implemented as a lighting effect function component 51 and can be connected to a power supply via the control main board 8. When the electric toothbrush is turned on, the lighting effect function component 51 can be controlled by the control main board 8 to operate, which can be applied according to a user's need.

The lighting effect function component 51 can be implemented as multiple therapeutic equivalents, such as red light therapy, blue light whitening or violet light sterilization, etc. In combination with the brush head 5, a comprehensive effect of protecting gums and cleaning teeth is achieved.

The power-consuming component can be implemented as a sensor and/or a photographic component. The internal environment and conditions of the oral cavity can be visually known via the sensor and the photographic element. It can even provide a direct understanding of conditions such as gum inflammation and tooth decay.

The specific embodiments mentioned above do not constitute a limitation on the protection scope of the present disclosure. Those skilled in the art should understand that various modifications, combinations, sub-combinations, and substitutions can be made according to design requirements and other factors. Any modifications, equivalent replacements, and improvements made within the spirit and principles of this disclosure shall be included within the protection scope of the present disclosure.

Those skilled in the art should understand that the embodiments of the present disclosure described above are for illustration only and do not limit the disclosure. The objectives of the present disclosure have been fully and effectively achieved. The functions and structural principles of the present disclosure have been shown and explained in the embodiments. Without departing from these principles, the embodiments of the present disclosure can be modified or varied in any way.

What is claimed is:

1. A conductive structure of a sonic motor, comprising:
a sonic motor body;
a hollow conductive motor shaft, one end of the hollow conductive motor shaft passing through an interior of the sonic motor body and being driven by the sonic motor body to perform high-frequency reciprocating vibrations with a certain amplitude, a segment of the hollow conductive motor shaft extending out from the sonic motor body to form an output segment, the output segment having a first electrical output terminal, the hollow conductive motor shaft having a first electrical input terminal;
a conductive assembly, the conductive assembly at least comprising a first conductive component, a second conductive component and an insulating mounting base, the first conductive component being installed on the output segment via the insulating mounting base to form a second electrical output terminal, one end of the second conductive component being connected to the first conductive component, another end of the second conductive component passing through the hollow conductive motor shaft and extending out from the hollow conductive motor shaft to form a second electrical input terminal; and
a mounting bracket, the mounting bracket being sleeved on the hollow conductive motor shaft and being at least circumferentially fixed relative to the hollow conductive motor shaft, the mounting bracket having a first mounting port at the first electrical output terminal for mounting and connecting a power-consuming component, the mounting bracket having a second mounting port at the second electrical output terminal for mounting and connecting the power-consuming component.

2. The conductive structure of the sonic motor according to claim 1, wherein the hollow conductive motor shaft further comprises a motor shaft wire, one end of the motor shaft wire is in electrical connection with the first electrical output terminal, and another end of the motor shaft wire is implemented as the first electrical input terminal.

3. The conductive structure of the sonic motor according to claim 2, wherein the motor shaft wire is wrapped with insulating plastic.

4. The conductive structure of the sonic motor according to claim 2, wherein a connection hole is formed on a side wall of the output segment, the one end of the motor shaft wire is connected to the hollow conductive motor shaft in a hidden way via the connection hole; the other end of the motor shaft wire extends within the hollow conductive motor shaft or extends outside the hollow conductive motor shaft.

5. The conductive structure of the sonic motor according to claim 2, further comprising a first conductive plate, the first conductive plate having a through hole, the hollow conductive motor shaft passing through the through hole and being in contact with the first conductive plate, the one end of the motor shaft wire being connected to the first conductive plate.

6. The conductive structure of the sonic motor according to claim 5, wherein the first conductive plate is further provided with a flexible contact portion in the through hole, the first conductive plate is electrical contact with the hollow conductive motor shaft via the flexible contact portion.

7. The conductive structure of the sonic motor according to claim 6, wherein two flexible contact portions are provided, and the two flexible contact portions are located on two sides of the hollow conductive motor shaft and are in contact with the hollow conductive motor shaft.

8. The conductive structure of the sonic motor according to claim 6, wherein the flexible contact portion is implemented as a metal wire or a strand of metal wires.

9. The conductive structure of the sonic motor according to claim 1, wherein the first conductive component is a first electrode, the second conductive component is a second wire; the first electrode is installed at a rear end of the output segment via the insulating mounting base, the second wire passes through the hollow conductive motor shaft and is connected to the first electrode, the second electrical input terminal is defined as an end of the second wire that is far away from the first electrode, the second electrical output terminal is defined as the first electrode.

10. The conductive structure of the sonic motor according to claim 1, further comprising an electrical connection assembly, the electrical connection assembly comprising a first electrical connector and a second electrical connector, the first electrical connector being detachably mounted at the first mounting port and being contact with the first electrical output terminal, the second electrical connector being detachably mounted at the second mounting port and being contact with the second electrical output terminal; the first electrical connector and the second electrical connector being connected to positive and negative electrodes of the power-consuming component through wires to provide power.

11. A brush head power supply structure for an electric toothbrush, comprising at least a brush head and the conductive structure of the sonic motor as claimed in claim 1, the power-consuming component being arranged in the brush head, a positive electrode and a negative electrode of the power-consuming component being correspondingly connected to the first electrical output terminal and the second electrical output terminal.

12. The brush head power supply structure for an electric toothbrush according to claim 11, further comprising a brush head handle, the brush head being arranged on the brush head handle, the brush head handle having an insertion cavity, an electrical connection assembly being provided in the insertion cavity; the electrical connection assembly comprising a first electrical connector and a second electrical connector, the first electrical connector and the second electrical connector being respectively connected to the positive electrode and the negative electrode of the power-consuming component through wires; the mounting bracket being inserted into the insertion cavity, the first electrical connector being detachably mounted at the first mounting port and being contact with the first electrical output terminal, the second electrical connector being detachably mounted at the second mounting port and being in contact with the second electrical output terminal.

13. The brush head power supply structure for an electric toothbrush according to claim 12, wherein the first electrical connector is conductive elastic plate, an elastic deformation portion is provided at a bottom of the conductive elastic plate, a guide slot is provided on the mounting bracket along an insertion direction, the first mounting port is located in the guide slot, the conductive elastic plate compresses the elastic deformation portion to allow the elastic deformation portion to be inserted into the first mounting port along the guide slot, the elastic deformation portion extends into the first mounting port and is in electrical contact with the first electrical output terminal.

14. The brush head power supply structure for an electric toothbrush according to claim 12, wherein the second electrical connector is a conductive probe, the conductive probe is inserted into the second mounting port and is in electrical contact with the first conductive component.

15. An electric toothbrush, comprising the brush head power supply structure for an electric toothbrush of claim 11, the first electrical input terminal and the second electrical input terminal are connected to a control main board or a battery of the electric toothbrush.

16. The electric toothbrush according to claim 15, wherein the power-consuming component is a lighting effect function component, the lighting effect function component comprises a light board and a light bead, the light board is installed in the brush head, the light board is electrically connected to the first electrical output terminal and the second electrical output terminal, the light bead is installed on the light board.

17. The electric toothbrush according to claim 16, wherein the light bead is implemented as one of a red light therapy bead, a blue light brightening bead or a violet light sterilization bead or any combination thereof.

18. The electric toothbrush according to claim 17, wherein bristles of the electric toothbrush are partially implemented as light-conducting bristles.

19. The electric toothbrush according to claim 18, wherein the light-conducting bristles are transparent bristles or bristles into which a light guide agent has been added during preparation.

20. The electric toothbrush according to claim 15, wherein the power-consuming component is implemented as a sensor and/or a photographic component.
